(19) United States
(12) Patent Application Publication
(10) Pub. No.: US 2022/0387511 A1
(43) Pub. Date: Dec. 8, 2022

(54) STEM CELL IMPREGNATED CORTICAL FIBERS

(71) Applicant: Zavation Medical Products, LLC, Flowood, MS (US)

(72) Inventor: Jeffrey Johnson, Flowood, MS (US)

(21) Appl. No.: 17/825,950

(22) Filed: May 26, 2022

Related U.S. Application Data
(60) Provisional application No. 63/193,153, filed on May 26, 2021.

(51) Int. Cl. A61K 35/28

(52) U.S. Cl. CPC A61K 35/28; A61K 2035/124

(57) ABSTRACT

A bone augmentation composition and a method for making the bone augmentation. The method including hydrating an allograft comprising cortical fibers with a cell culture media, seeding the hydrated allograft with a solution of human stem cells, and culturing the stem-cell seeded allograft to grow a population of the stem cells in the seeded graft prior to freezing the bone augmentation composition for storage.

### CROSS-REFERENCE TO RELATED APPLICATION

This application claims the benefit of U.S. Provisional Application No. 63/193,153, filed on May 26, 2021, the entire contents of which are incorporated herein by reference.

### BACKGROUND OF THE INVENTION

### Field of the Invention

The present invention relates to bone augmentation materials specifically including bone allografts.

### Description of the Related Art

Bone grafting is a surgical procedure that replaces missing bone and/or repairs bone fractures. Bone grafts may be allograft (cadaveric bone e.g., from a bone bank), autologous (i.e., bone harvested from the patient's own body, for example from the iliac crest), or synthetic. Most bone grafts are expected to be resorbed and replaced as the natural bone heals over time.

Bone grafts can promote osteoconduction (guiding the reparative growth of the natural bone), osteoinduction (encouraging undifferentiated cells to become active osteoblasts), and/or osteogenesis (living bone cells in the graft material contributing to bone remodeling). Bone grafts are considered osteogenic if they contain viable cells that are capable of bone regeneration, which is advantageous for bone healing. Osteogenesis occurs with autografts. Autografts are considered osteogenic because they contain a high number of bone forming cells.

In general, multiple bone injuries can lead to the need for bone allografts. Bone allografts substantially increase the rate of healing but the healing process is usually still long and arduous. For this reason bone allografts are an active area of research.

US Pat. Appl. Publ. No. 2020/0222590A1, which is hereby incorporated by reference in its entirety, describes with a scaffold that can provide bone allograft material along with cells or therapeutic molecules.

US Pat. Appl. Publ. No. 2017/0197010, which is hereby incorporated by reference in its entirety, describes a composite bone graft with a synthetic bone portion and an allograft portion.

U.S. Pat. No. 10,172,651, which is hereby incorporated by reference in its entirety, describes an apparatus with a cortical bone surface sandwiched between two perforated demineralized bone matrix surfaces.

U.S. Pat. No. 10,548,923, which is hereby incorporated by reference in its entirety, describes applying an ionic force charge agent to allograft bone material and thus enhancing growth factor binding.

US Pat. Appl. Publ. No. 20110118850, which is hereby incorporated by reference in its entirety, describes applying lysed bone material to various bone implants in order to apply growth factors and bioactive materials.

US Pat. Appl. Publ. No. 20130101637, which is hereby incorporated by reference in its entirety, describes bone cages that can be used to apply living cells and tissues as well as biologically active molecules.

U.S. Pat. No. 8,834,928, which is hereby incorporated by reference in its entirety, describes tissue repair implants where at least one population of viable tissue-genic cells adhere and reside in a growth conductive matrix.

Mechanical characterization of bone allografts enriched with mesenchymal cells: theory and applications by Jose Luis Diaz Leon, Ramon Rodriguez, and Raul Lesso Arroyo in Science and Engineering, Vol 98, © 2017 WIT Press, pp. 240-247, which is hereby incorporated by reference in its entirety, mechanically characterizes cancellous and cortical/cancellous allograft tissue chips along with flexure and pure shear testing of cortical allograft strips.

Allogenic bone graft enriched by periosteal stem cell and growth factors for Osteogenesis in critical size defect in rabbit model: histopathological and radiological evaluation by Hadi Hassibi, Alireza Farsinejad, Shahriar Dabiri et al and published in Iran J. Pathol. 2020; 15(3): pp. 205-216, which is hereby incorporated by reference in its entirety, discusses the effects of periosteal stem cell enrichment of allogenic bone grafts in a rabbit model.

Tissue engineering using a combined cell sheet technology and scaffolding approach by Irina M. Zurina, Viktoria S. Presniakova, and Denis V. Butnaru et al and published in Acta Biomaterialia 113 (2020) pp. 63-83, which is hereby incorporated by reference in its entirety, describes various methods of combining cell sheets and scaffolds.

Bone grafts and biomaterials substitutes for bone defect repair. A review by Wenhao Wang, and Kelvin W. K. Yeung and published in Bioactive Materials 2 (2017) pp. 224-247, which is hereby incorporated by reference in its entirety, reviews bone grafts, bone graft substitutes along with additions of growth factors and bioinorganic ions.

The effect of mesenchymal stem cell sheets on structural allograft healing of critical sized femoral defects in mice by Teng Long, Zhenan Zhu, Hani A. Awad et al and published in Biomaterials 35 (2014) pp. 2752-2759, which is hereby incorporated by reference in its entirety, describes the use of structural allografts wrapped with mesenchymal stem/progenitor cell sheets in a mouse model.

U.S. Pat. No. 9,192,695, which is hereby incorporated by reference in its entirety, describes digesting tissue to form a cell suspension with MSCs along with unwanted cells and allowing this suspension to adhere to a bone substrate.

U.S. Pat. No. 5,733,542, which is hereby incorporated by reference in its entirety, describes methods and preparations of administering a bone marrow graft with culturally expanded mesenchymal stem cells.

U.S. Pat. No. 6,355,239, which is hereby incorporated by reference in its entirety, describes methods and devices using non-autologous mesenchymal stem cells in treating and regenerating connective tissue as well as enhancing bone marrow engraftment.

U.S. Pat. No. 6,541,024, which is hereby incorporated by reference in its entirety, describes delivery of isolated, culture expanded human mesenchymal stem cells, freshly aspirated bone marrow either alone or together in a carrier material or matrix.

U.S. Pat. No. 9,511,093, which is hereby incorporated by reference in its entirety, describes an osteogenic composition comprising mesenchymal stem cells pre-cultured in the presence of an agent that accelerates canonical Wnt signaling.

U.S. Pat. No. 9,539,286, which is hereby incorporated by reference in its entirety, describes multiple examples of bone grafts and constructs containing stem cells.

### SUMMARY OF THE INVENTION

In one embodiment there is disclosed a method for making a bone augmentation composition comprising hydrating an allograft comprising cortical fibers with a cell culture media, seeding the hydrated allograft with a solution of human stem cells, and culturing the stem-cell seeded allograft to grow a population of the stem cells in the seeded graft prior to freezing the bone augmentation composition for storage.

In an embodiment a bone augmentation composition comprising stem cells, a cell culture medium, and an allograft comprising bone fibers wherein the allograft comprising the stem cells and the cell culture medium, after freezing and thawing, has a density of 5×10⁴ cells/ml to 1×10⁶ cells/ml as counted by an Alamar Blue Assay.

It is to be understood that both the foregoing general description of the invention and the following detailed description are exemplary, but not restrictive of the invention.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing, wherein:

FIG. 1 is a diagram showing one embodiment of the disclosed method;

FIG. 2 is a diagram showing another embodiment of the disclosed method;

FIG. 3 is a diagram showing still another embodiment of the disclosed method;

FIG. 4A is a graph showing analysis of viable cells seeded on cortical fiber bone allografts using cell detachment;

FIG. 4B is a graph showing analysis of viable cells seeded on cortical fiber bone allografts using an Alamar Blue Assay;

FIG. 5 is a graph showing the adherence of viable cells on cortical fiber bone allografts over five days with static or dynamic seeding:

FIG. 6 is a graph showing initial adherent cell concentrations with varying cell seeding concentrations;

FIG. 7 is a graph showing adherent cell number over five days of culture using a low or high seeding concentration; and

FIG. 8 is a graph showing viable adherent cell concentration of seeding grafts pre-freeze and post thaw after varying incubation times.

### DETAILED DESCRIPTION

The present invention provides a bone augmentation composition and a method for making the bone augmentation. The bone augmentation composition includes a mixture of human mesenchymal stem cells, cortical fibers, and an optional stem cell culture medium. Methods of making the bone augmentation composition and methods of preserving the bone augmentation composition for later use are described herein.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The terms “comprises,” “comprising.” “includes,” “including,” “having” and their conjugates mean “including but not limited to”. This term encompasses the terms “consisting of” and “consisting essentially or”.

The phrase “consisting essentially of” means that the composition or method may include additional ingredients and/or steps, but only if the additional ingredients and/or steps do not materially alter the basic and novel characteristics of the claimed composition or method.

A “subject,” as used herein, can be any animal, and may also be referred to as the patient. Preferably the subject is a vertebrate animal, and more preferably the subject is a mammal, such as a research animal (e.g., a mouse or rat) or a domesticated farm animal (e.g., cow, horse, pig) or pet (e.g., dog, cat). In some embodiments, the subject is a human.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

“Biocompatible” as used herein, refers to any material that does not cause injury or death to a subject or induce an adverse reaction in a subject when placed in contact with the subject's tissues. Adverse reactions include for example inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

As used herein, “treatment” means any manner in which the symptoms of a defect, condition, disorder, or disease, or any other indication, are ameliorated or otherwise beneficially altered.

All ranges in the below are open as opposed to closed ended such that the range 1 to 10 includes the range 1.1 to 9.9 as well as the range 1 to 10.

Manufactured cortical fibers are made from bone fibers. When used for orthopedic repair, these manufactured products are typically set into bone as a replacement for missing bone. Manufactured cortical fiber products can stimulate bone growth and serve as replacement bone especially when a patient's bone lacks structure from bone loss or injury. The manufactured cortical fiber products often have a moldable, dough-like texture and consistency for shaping and conforming to an injury site where they are to be applied. The manufactured cortical fiber products often are made from entangled fibers.

In general, cortical fibers are obtained from a natural bone source by removing marrow and fat, and then demineralizing and grinding the cortical bone. In one embodiment, bone can be cleaned of soft tissues, bone marrow can be removed using warm saline water and agitation, and the bone can then be wrapped in cotton towels and sealed in plastic bags and rapidly frozen in liquid nitrogen vapor. The refined bone can be freeze-dried for 5 days with a freeze dryer condenser at −50° C. to −60° C. on shelves at −30° C. to −20° C. Shelves can be heated to 25° C. before freeze dried bone is removed. Freeze dried bone can then be ground incrementally and sieved.

In another embodiment, preprocessed ilium tissue can be generated by exposing ilium tissue to a bioburden reducer. The preprocessed ilium tissue can then be soaked three to four times with agitation in a buffered isotonic solution, debrided of all soft tissue and cut for example into approximately 3×3 cm strips which can then be milled into pellets of approximately 2 mm in diameter or less. Pellets can be rinsed with phosphate buffered saline (PBS) and then acetic acid solution for five minutes and then rinsed again with PBS until the pH of the rinseate is near a physiological pH. Alternatively, the bone pellets can be rinsed with HCl or citric acid for demineralization and cleaning, or demineralization can take place using citric acid perfusion into bone pores to decrease demineralization time and ensure uniform calcium removal.

Suitable cortical fiber products suitable for the present invention include ENHANCE® demineralized cortical fibers from Con Med (www.conmed.com, 11311 Concept Blvd. Largo, Fla. 33773, 727-392-6464), AlloFuse® Cortical Fibers from AlloSource, (www.allosource.org, 6278 S Troy Cir Centennial, Colo. 80111, 800-557-3587). DBM Fiber from Origin Biologics, (www.originbio.com, 6635 S. Eastern Avenue Suite 100 Las Vegas, Nev. 89119, 702-790-7015), cortical fibers from Stability Biologics (www.stabilitybio.com, 2026 Fransworth Dr., Nashville, Tenn., 37025, 855-267-5551), Incite® Cortical Fibers from Spineology (www.spineology.com, 7800 3^{rd} Street North. Suite 600 St. Paul, Minn. 55128-5455, 888-377-4633) and others.

Human mesenchymal stem cell isolation and culture expansion is described in U.S. Pat. No. 5,486,359 which is hereby incorporated by reference in its entirety.

Isolation culturing and transplantation of stem cells is discussed in What Stem Cells Should be Used for Transplantation in Fetal Diagn. Ther (2004) 19: pp. 2-8 which is hereby incorporated by reference in its entirety; Fetal stem cells in Best Practice & Research Clinical Obstetrics Gynaecology (2004) 18: pp. 853-875 which is hereby incorporated by reference in its entirety; Characterization of cells with osteogenic potential from human marrow in Bone 1992 13: pp. 81-88 which is hereby incorporated by reference in its entirety; Mesenchymal Stem Cells: Cell Biology and Potential Use in Therapy Basic & Clinical Pharmacology & Toxicology (2004) 95: pp. 209-214 which is hereby incorporated by reference in its entirety; and Genetic Modification of Stem Cells to Enhance Bone Repair in Ann Biomed Eng. (2004) 32: pp. 136-147 which is hereby incorporated by reference in its entirety.

Homogeneous human mesenchymal stem cells can be isolated by positively selecting adherent marrow or periosteal cells which lack markers associated with either hematopoietic cells or differentiated mesenchymal cells. In one embodiment, a tissue specimen containing mesenchymal stem cells such as bone marrow, embryonic yolk sac, placenta, umbilical cord, fetal or adolescent skin and blood can be added to a medium containing factors that stimulate mesenchymal stem cell growth without differentiation and allow for the selective adherence of only mesenchymal stem cells to a substrate surface. This isolated human mesenchymal stem cell mixture can then be cultured, and the non-adherent matter removed from the substrate.

The isolated human mesenchymal stem cells can then be culture expanded by adding cells to a medium which contains factors that stimulate mesenchymal stem cell growth without differentiation and allow selective adherence of only the mesenchymal stem cells to the substrate surface, culturing the medium, removing the non-adherent matter from the substrate surface by replacing medium with fresh medium and allowing the isolated adherent mesenchymal stem cells to culture-expand.

In one example, 3700 mg/I of sodium bicarbonate, and 10 ml/l of 100× antibiotic-antimycotic containing 10,000 units of penicillin (base), 10,000 μg of streptomycin (base) and 25 μg of amphotericin B/ml utilizing penicillin G (sodium salt), streptomycin sulfate, and amphotericin B as FUNGIZONE® in 0.85% saline can be added to commercially available Dulbecco's Modified Eagle's Medium. When ready to be used, fetal bovine serum can be added until a final volume of 10% serum is reached. A bone growth medium such as for example BGJ_{b} medium (Gibico, Grand Island N.Y.), F-12 Nutrient Mixture (Gibico, Grand Island N.Y.), with 10% by volume fetal bovine serum can also be used.

In one embodiment, marrow can be added to one of the above media and vortexed to form a dispersion which can be centrifuged to separate the marrow cells which can then be dissociated into single cells by passing the medium containing the marrow cells through syringes fitted with 16, 18, and/or 20 gauge needles in series. The resulting suspension can then be plated into 100 mm dishes. Marrow cells can be allowed to grow and adhere to the surface of the Petri dishes for example from one to seven days. Non-adherent cells can be removed by replacing the original medium with fresh complete medium. This process can be repeated until the culture dishes become confluent. Cells can be detached using for example trypsin and (ethylenedinitrilo)tetraacetic acid EDTA.

Suitable human mesenchymal stem cell products for the present invention include Human Umbilical Mesenchymal Stem Cell Pellet from ScienCell (www.sciencelonline.com, 1610 Faraday Ave, Carlsbad Calif., 92008, 877-602-8549), HighQC™ Human Mesenchymal Bone Marrow (Adult) Derived Stem Cell from Accegen (www.Accegen.com 277 Fairfield Rd, Fairfield, N.J. 07004, 862-686-2696), Bone Marrow Mesenchymal Stem Cells, Frozen from stemexpress (www.stemexpress.com 1743 Creekside Drive, Suite 200, Folsom, Calif. 95630, 530-626-7000) and others.

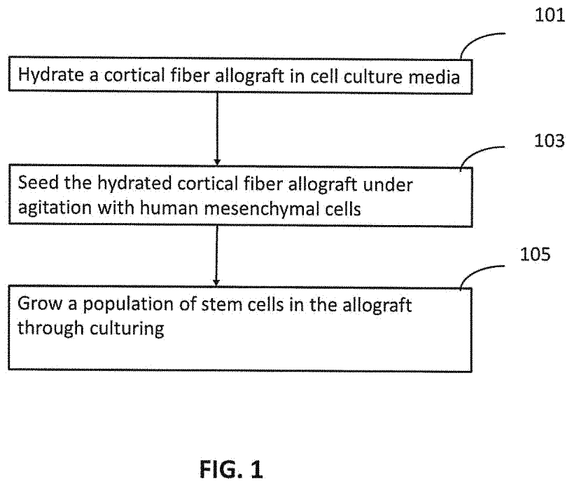

In one embodiment of the invention, as shown at steps 101, 103, and 105 of FIG. 1, an allograft comprising cortical fibers can be hydrated with a cell culture media. The hydrated allograft can be seeded under agitation with a solution of human mesenchymal stem cells derived from a source other than the patient being treated. A population of stem cells can be grown in the seeded allograft through culturing.

More specifically, there is provided a method for making a bone augmentation composition, comprising: a) hydrating an allograft comprising cortical fibers with a cell culture media, b) seeding the hydrated allograft with human mesenchymal stem cell solution derived from a source other than the patient being treated, wherein the seeding optionally comprises a dynamic seeding where the hydrated allograft is agitated at least during application of the stem cell solution to the hydrated allograft, c) culturing the stem-cell seeded allograft for a predetermined time (or a limited time to reduce stem cell attachment to the allograft), d) freezing the stem-cell seeded allograft containing the cell culture media with a cryopreservation solution, e) and after cryopreservation, thawing the stem-cell seeded allograft containing the cell culture media to provide the bone augmentation composition for application to a bone treatment site. In one embodiment, a surviving stem cell population in the bone augmentation composition after thawing is between 50% and 90% of an initial stem cell population in the bone augmentation composition prior to freezing. In one embodiment, the seeding, the limited time of culturing, and the freezing produce, after thawing, a surviving stem cell population in the bone augmentation composition that is between 50% and 90% of an initial stem cell population in the bone augmentation composition prior to freezing.

In one embodiment, reducing attachment of the stem cells to the allograft fibers promotes a higher surviving stem cell population. In one embodiment, a prolonged time (as opposed to a limited time of culturing generally less than 5 hrs) reduces the surviving stem cell population. This observation is considered to be caused because the prolonged times cause more adherence of the stem cells to the bone allografts and cell death by freezing when the stem cells are so adhered.

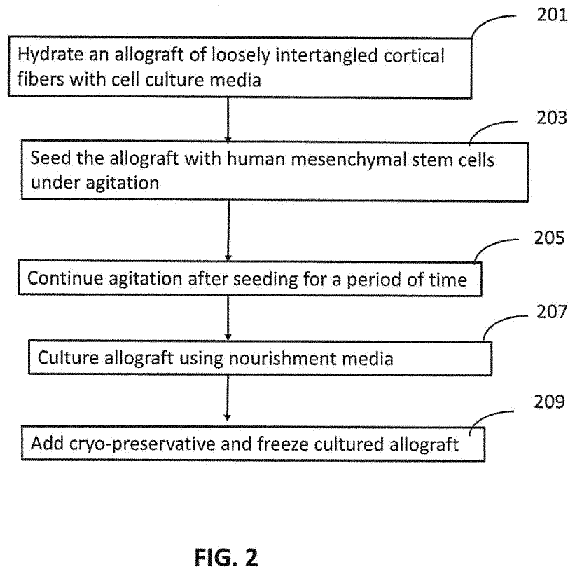

In one embodiment, as outlined in FIG. 1 and as shown at steps 201, 203, 207, 207, and 209 of FIG. 2, an allograft comprising a loose, intertangled mixture of cortical fibers can be hydrated with cell culture media; the hydrated allograft can be seeded (under agitation) with a solution of human mesenchymal stem cells; and the agitation continued after seeding is completed. As used herein, “loose” in reference to a mixture of cortical fibers means that the cortical fibers are not in a pellet form and contain air space between many of the individual fibers. In one embodiment, the seeded allograft can be cultured with a benign detachment solution (i.e., a first solution) such as for example a TryPLE solution (available from ThermoFisher Scientific) or an Accutase solution (available from Stem Cell Technologies to detach stem cells from the seeded graft. In one embodiment, the seeded allograft can be cultured with a nourishment solution (i.e., a second solution) such as a RoosterNourish solution (available from Rooster Bio) or StemSpan (available from Stem Cell Technologies). Once the culture ends, the seeded allograft can be frozen at −80° C. with a cryo-preservative such as for example dimethyl sulfoxide DMSO or can be frozen at other temperatures suitable for the cryo-preservative chosen.

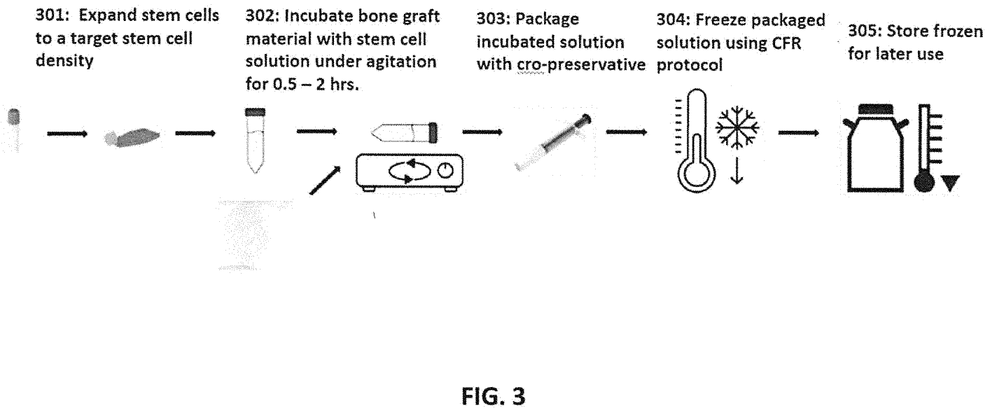

FIG. 3 is a diagram showing an overall processing method. At 301, stem cells from a working cell bank (WCB) are expanded to a target stem cell density (cells/ml) for addition to a bone graft. At 302, a bone graft material (from a pellet stock or from a loose fiber stock) is incubated with a stem cell solution optionally containing a cell culture medium, and the bone graft and stem cell solution is agitated for 0.5 to 2 hrs. At 303, the incubated solution is packaged into a contained with a cryo-preservative. At 304, freeze packaged solution under a controlled rate (CRF) protocol such as for example at a freezing rate of −1° C./min in the cryo-preservative. At 305, the packaged solution is frozen for later use. at liquid nitrogen or at −80° C.

In one embodiment, agitation of the seeded allograft and/or the culturing of the seeded allograft can occur for a total time that minimizes stem cell attachment to the allograft. In this embodiment, agitation and/or culturing can continue for about 0.5 hours, or for about 1 hour, or for about 1.5 hours, or for 2 about hours, or for about 2.5 hours, or for about 3 hours, or for about 3.5 hours, or for about 4 hours, or for about 4.5 hours, or for about 5 hours. In various embodiments, the agitation and/or the culturing of the seeded allograft occurs for 0.5 to 1 hours, or for 1 to 1.5 hours, or for 1.5 to 2 hours, or for 2 to 2.5 hours, or for 2.5 to 3 hours, or for 3 to 3.5 hours, or for 3.5 to 4 hours, or for 4 to 4.5 hours, or for 4.5 to 5 hours, or for 0.5 to 5 hours, or for 1 to 5 hours, or for 1.5 to 5 hours, or for 2 to 5 hours, or for 2.5 to 5 hours, or for 3 to 5 hours, or for 3.5 to 5 hours, or for 4 to 5 hours, or for 4.5 to 5 hours, or possibly longer. The agitation can occur by having the seeded allograft in a container which is reciprocally shaken, ultrasonically shaken, or stirred.

In various embodiments, the stem cell seeding itself can take place under a variety of agitations included but not limited to those described above, and at an agitation of 50 to 500 rpms, an agitation of 60 to 500 rpms, an agitation of 70 to 500 rpms, an agitation of 80 to 500 rpms, or an agitation of 90 to 500 rpms, and at any intervening rpm range, or higher or lower.

In one embodiment, the hydrated allograft can be seeded with a stem cell solution having a stem cell density of about 2×10⁵ cells/ml. In another embodiment, the hydrated allograft can be seeded with a stem cell solution having a stem cell density of about 3×10⁵ cells/ml. In another embodiment, the hydrated allograft can be seeded with a stem cell solution having a stem cell density of about 4×10⁵ cells/ml. In one embodiment, the hydrated allograft can be seeded with a stem cell solution having a stem cell density of about 5×10⁵ cells/ml. In another embodiment, the hydrated allograft can be seeded with a stem cell solution having a stem cell density of about 6×10⁵ cells/ml. In still another embodiment, the hydrated allograft can be seeded with a stem cell solution having a stem cell density of about 7×10⁵ cells/ml. In one embodiment, the hydrated allograft can be seeded with a stem cell solution having a stem cell density of about 8×10⁵ cells/ml. In one embodiment, the hydrated allograft can be seeded with a stem cell solution having a stem cell density ranging from about 2×10 to 8×10⁵ cells/ml. In various embodiments, the hydrated allograft can be seeded with a stem cell solution having a stem cell density of 3×10⁵ to 8×10⁵ cells/ml, or having a stem cell density of 4×10⁵ to 8×10⁵ cells/ml, or having a stem cell density of 5×10⁵ to 8×10⁵ cells/ml, or having a stem cell density of 6×10⁵ to 8×10⁵ cells/ml, or having a stem cell density of 7×10⁵ to 8×10⁵ cells/ml, or having a stem cell density of 2×10⁵ to 3×10⁵ cells/ml, or having a stem cell density of 3×10⁵ to 4×10⁵ cells/ml, having a stem cell density of 4×10⁵ to 5×10⁵ cells/ml, having a stem cell density of 5×10⁵ to 6×10⁵ cells/ml, having a stem cell density of 6×10⁵ to 7×10⁵ cells/ml, or having a stem cell density of 7×10⁵ to 8×10⁵ cells/ml.

In one embodiment, the stem cell incubated bone allograft solution has a cryo-preservative added to it such a dimethyl sulfoxide DMSO. The DMSO concentration can be less than about 20 weight percent, or less than about 10 weight percent, or less than about 5 weight percent, or less than about 2 weight percent.

In one embodiment, the controlled freezing rate (CRF) of the incubated solution of stem cells, cell culture medium, bone allograft, and cryo-preservative is one embodiment about −1° C./min. Other CRFs can be used ranging from about −0.2 to −10° C./min, and intermittent freezing rates. In this embodiment of the invention, one way to produce a constant freezing rate is to bring the temperature of the incubated solution near freezing, and then vary the temperature of the freezing chamber in accordance with the time/temperature shown in Table I below:

TABLE 1

Wait at Chamber = 0.0 C. until Sample = 4.0 C.
Ramp 1.0 C./min until Sample = −4.0 C.
Ramp 90.0 C./min until Chamber = −70.0 C.
Ramp 10.0 C./min until Chamber = −40.0 C.
Ramp 8.0 C./min until Chamber = −20.0 C.
Ramp 1.0 C./min until Chamber = −80.0 C.
End

In one embodiment, the population of stem cells in the seeded allograft after culturing and after being thawed can range from about 5×10⁴ to 1×10⁶ cells/ml. In one embodiment, the population of stem cells in the seeded allograft after culturing and after being thawed can be about 5×10⁴. In another embodiment, the population of stem cells in the seeded allograft after culturing and after being thawed can be about 6×10⁴ cells/ml. In another embodiment, the population of stem cells in the seeded allograft after culturing and after being thawed can be about 7×10⁴ cells/ml. In one embodiment, the population of stem cells in the seeded allograft after culturing and after being thawed can be about 8×10⁴ cells/ml. In another embodiment, the population of stem cells in the seeded allograft can be about 9×10⁴ cells/ml. In one embodiment, the population of stem cells in the seeded allograft after culturing and after being thawed can be about 1×10⁵ cells/ml. In another embodiment, the population of stem cells in the seeded allograft after culturing and after being thawed can be about 2×10⁵ cells/ml. In one embodiment, the population of stem cells in the seeded allograft after culturing and after being thawed can be about 3×10⁵ cells/ml. In one embodiment, the population of stem cells in the seeded allograft after culturing and after being thawed can be about 4×10⁵ cells/ml. In one embodiment, the population of stem cells in the seeded allograft after culturing and after being thawed can be about 5×10⁵ cells/ml. In another embodiment, the population of stem cells in the seeded allograft after culturing and after being thawed can be about 6×10⁵ cells/ml. In one embodiment, the population of stem cells in the seeded allograft after culturing and after being thawed can be about 7×10⁵ cells/ml. In another embodiment, the population of stem cells in the seeded allograft after culturing and after being thawed can be about 8×10⁵ cells/ml. In one embodiment, the population of stem cells in the seeded allograft c after culturing and after being thawed an be about 9×10⁵ cells/ml. In one embodiment, the population of stem cells in the seeded allograft after culturing and after being thawed can be about 1×10⁶ cells/ml. In one embodiment, the population of stem cells in the seeded allograft after culturing and after being thawed can be about 6×10⁴ to 1×10⁶ cells/ml. In various embodiments, the population of stem cells in the seeded allograft after culturing and after being thawed can range from 7×10⁴ to 1×10⁶ cells/ml, or can range from 8×10⁴ to 1×10⁶ cells/ml, or can range from e 9×10⁴ to 1×10⁶ cells/ml or In an embodiment the population of stem cells in the seeded allograft can range from 1×10⁵ to 1×10⁶ cells/ml, or can range from 2×10⁵ to 1×10⁶ cells/ml, or can range from 3×10⁵ to 1×10⁶ cells/ml, or can range from 4×10⁵ to 1×10⁶ cells/ml, or can range from 5×10⁵ to 1×10⁶ cells/ml, or can range from 6×10⁵ to 1×10⁶ cells/ml, or can range from 7×10⁵ to 1×10⁶ cells/ml, or can range from 8×10⁵ to 1×10⁶ cells/ml, or can range from 9×10⁵ to 1×10⁶ cells/ml, or can range from 5×10⁴ to 6×10⁴ cells/ml, can be 6×10⁴ to 7×10⁴ cells/ml, or can range from 7×10⁴ to 8×10⁴ cells/ml, can range from 8×10⁴ to 9×10⁴ cells/ml, or can range from 1×10⁵ to 2×10 cells/ml, or can range from 2×10⁵ to 3×10⁵ cells/ml, or can range from 3×10⁵ to 4×10⁵ cells/ml, or can range from 4×10⁵ to 5×10⁵ cells/ml, or can 5 range from ×10⁵ to 6×10⁵ cells/ml, or c range from an range from 6×10⁵ to 7×10⁵ cells/ml, or can range from 7×10⁵ to 8×10⁵ cells/ml, or can 8×10⁵ to 9×10⁵ cells/ml, or can range from 9×10⁵ to 1×10⁶ cells/ml, or higher.

In various embodiments, pre-incubated grafts range in a volume from 0.1 to 5 cc, and any intervening range, or higher or lower can be used.

While the embodiments have been described in connection with the various embodiments and figures above and will be described with the various examples below, it is to be understood that other similar embodiments may be used or modifications and additions may be made to the described embodiment.

### Example 1

2.5 cc cortical fiber grafts from Origin Biologics were cut into 7-8 smaller sections to allow for multiple tests from a single sample. Graft sections were either pre-hydrated with RoosterNourish Cell culture media for 24 hrs or seeded dry. Graft sections were directly inoculated with hMSCs by pipetting 300 μl 2×10⁶ cells/ml solution onto either pre-hydrated or dry graft sections. After 1 h incubation at 37° C., grafts were incubated in TrypLE Select cell detachment solution in order to detach cells from the grafts.

Additional seeded graft sections and unseeded controls were then transferred to 6-well plates and cultured in RoosterNourish for 6 days. After 6 days of incubation, graft sections were incubated with 10% Alamar Blue solution in RoosterNourish for 1 hr, then a 100 ul aliquot of Alamar Blue solution was transferred to a clear bottom 96 well plate. Samples were analyzed for fluorescence using a Biotek plate reader.

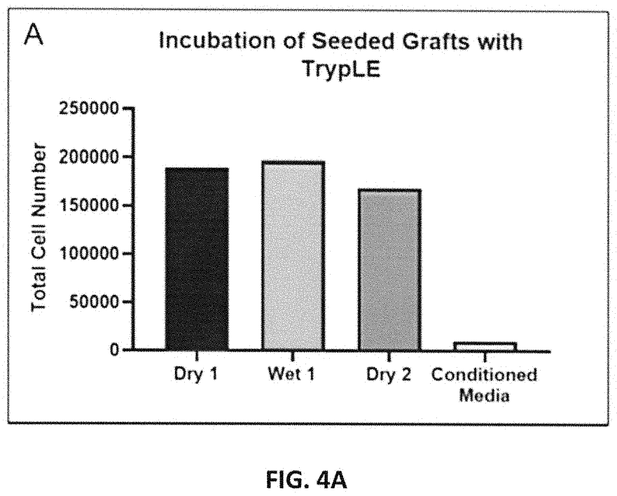

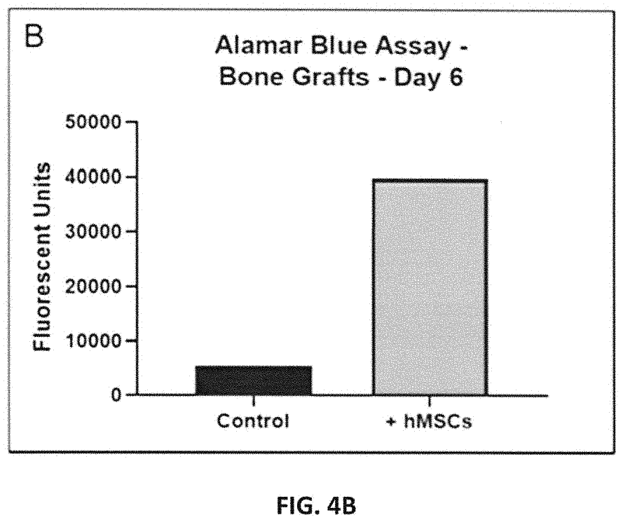

Analysis of TrypLE solution after 15 min incubation with seeded grafts using the NC-200 cell counter yielded a total cell number of around 2×10⁵ cells/ml. See FIG. 4A. Analysis of the media in which the grafts (without seeding) were incubated in RoosterNourish yielded a negligible cell number. For the grafts sections incubated in RoosterNourish for 6 days, a seeded graft section yielded a positive fluorescent signal, while an unseeded graft section did yielded only a minimal fluorescent signal. See FIG. 4B.

### Example 2

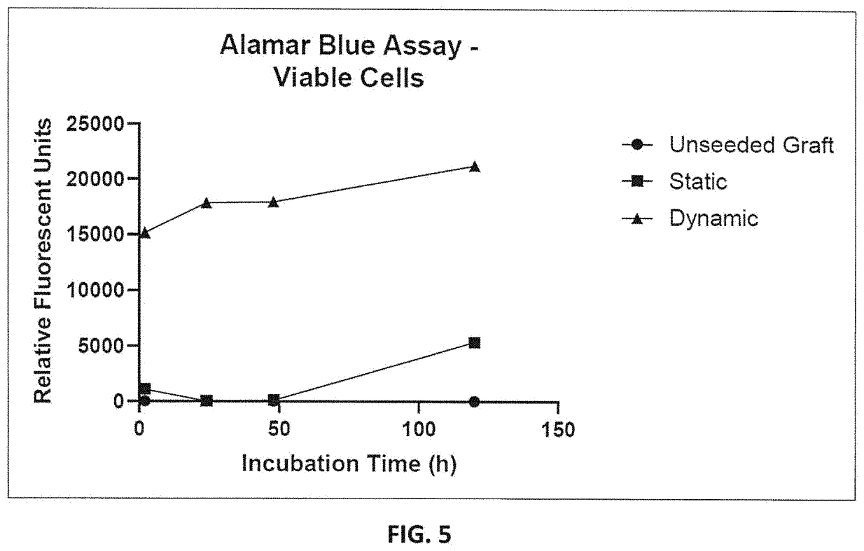

2.5 cc grafts from Origin Biologics were cut into 8 sections. A population of HMSCs were pre-stained with CellTrace Oregon Green 488 dye—a cell tracking dye that fluorescently stains the cell cytoplasm. Origin Biologics labeled hMSCs were seeded onto graft sections, then the seeded grafts were cultured in RoosterNourish. Fluorescent microscopy images were taken at 24, 28, and 120 hrs of post-seeding. Dynamically seeded graft sections were incubated in 10 ml of a 6.7×10⁵ cells/ml solution on a shaker plate at 125 rpm for 2 hr at room temperature. For static seeding, 300 μl of a 1×10⁶ cells/ml solution was added directly to each graft section, and the statically seeded solutions were then incubated at 37° C. for 1 hr. After seeding, all graft sections were either immediately analyzed for adherent viable cell number using the Alamar Blue assay or cultured in 8 ml Rooster Nourish for varying lengths of time before analysis using Alamar Blue. Initial adherent cell density on the dynamically seeded grafts was very high, and adherent cell density remained consistent over the five-day time course. See FIG. 5. Statically seeded grafts had comparatively low initial adherent cell density that did not reach the initial levels of the dynamically seeded grafts even after five days. See FIG. 5.

### Example 3

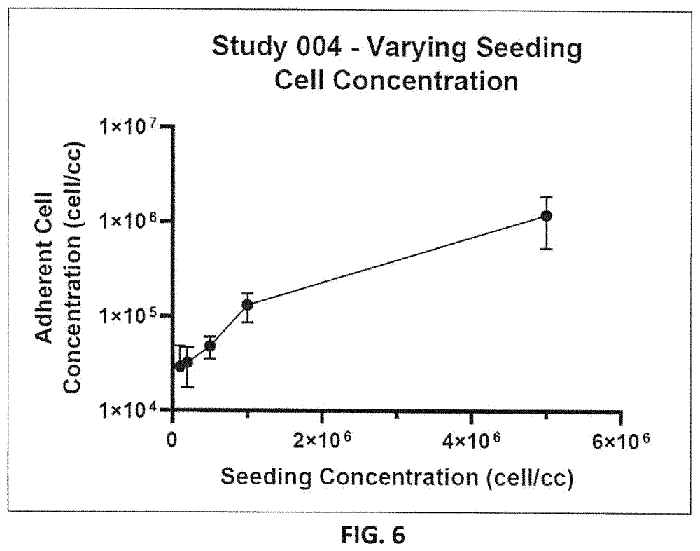

Cell seeding concentrations were varied from 1×10⁵ to 5×10⁶ cells/ml. Bone grafts from Origin Biologics were incubated in the cell solutions for 2 hours on a shaker plate at 125 rpm at RT. Immediately after seeding, the Alamar Blue assay was used to analyze initial adherent cell density, and a standard curve was used to determine absolute cell concentration. See FIG. 6.

### Example 4

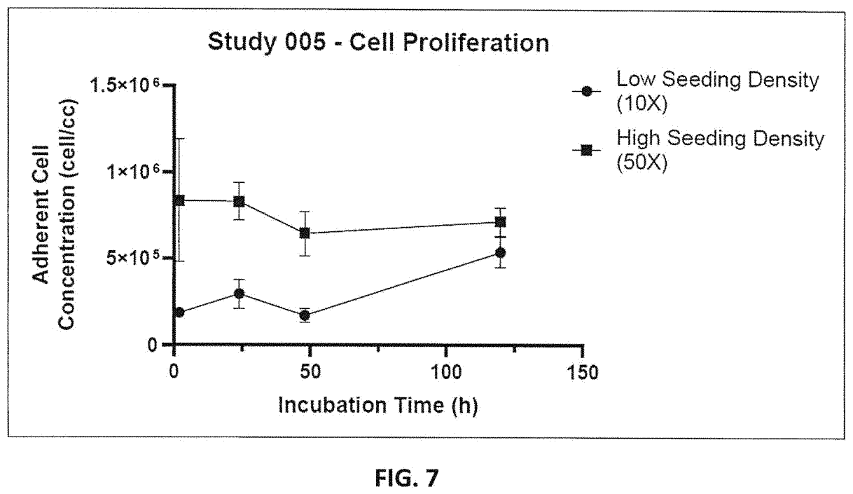

Graft sections from Origin Biologics were incubated with either 1×10⁶ cell/ml or 5×10⁶ cell/ml solution for 2 hr on a shaker plate at 125 rpm at RT, and then cultured in RoosterNourish growth media for up to 120 hrs. The Alamar Blue assay was used to quantify adherent cell number after 2 hrs, 24 hrs, 48 hrs, and 120 hrs of culturing. The high seeding concentration initially saturated the adherent cell density, and no change in cell number was observed over the five days of culture. See FIG. 7. For initial low seeding concentrations, the cell number remained consistent over the first 48 hrs of culture but showed a large increase in cell number at the 5 day time point, See FIG. 7.

### Example 5

Graft sections from Origin Biologics were incubated with 2×10⁶ cell/ml solution for 1-2 hour at RT on the shaker plate at 125 rpm then incubated for up to an additional time for total incubation times of 1 hr, 2 hrs, 4 hrs, and 24 hrs. Half of the samples were analyzed using Alamar Blue to determine pre-freeze adherent cell concentration, while the other half was incubated in a mixture of 10% dimethyl sulfoxide DMSO for 30 mins before cryopreservation.

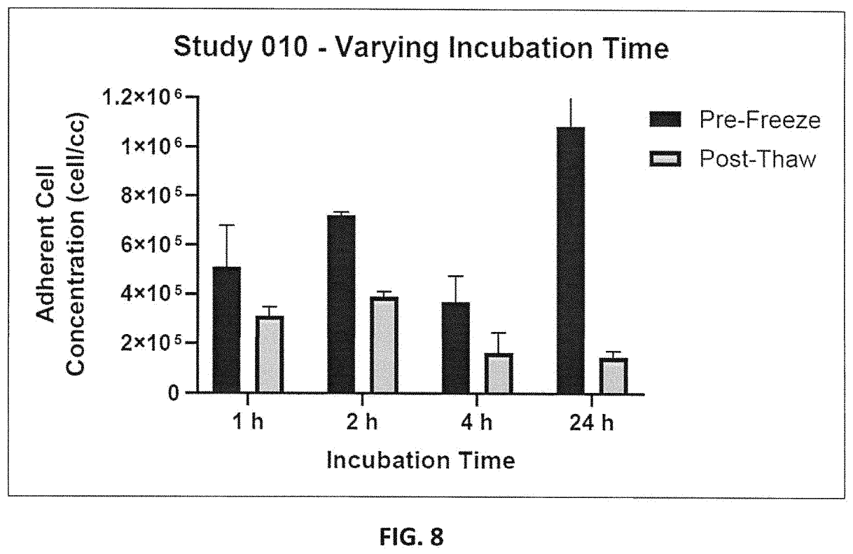

Frozen grafts were stored at −80° C. for 5 days, then were thawed in a 37° C. water bath for 10 mins, washed 3 times with PBS, then analyzed using Alamar Blue to determine post-thaw viable adherent cell concentration. Two standard curves one using cells used to seed the grafts to calculate pre-freeze cell density and one using cells that were cryopreserved in solution then thawed with seeded grafts to calculate post-thaw cell density. This process accounts for potential differences in the metabolism of freshly thawed cells as the Alamar Blue assay measures cell metabolism. The post-thaw viable adherent cell concentration peaked around 3.7×10¹ cell/mi (observed for 1 h and 2 h incubation times). See FIG. 8.

### STATEMENTS OF THE INVENTION

The examples and descriptions above show that the present invention is manifest in a variety of different embodiments as in the numbered statements below.

Statement 1. A method for making a bone augmentation composition, comprising: hydrating an allograft comprising cortical fibers with a cell culture media; seeding the hydrated allograft with human mesenchymal stem cell solution derived from a source other than the patient being treated; culturing the stem-cell seeded allograft for a predetermined time (or a limited time to reduce stem cell attachment to the allograft), freezing the stem-cell seeded allograft containing the cell culture media with a cryopreservation solution; and after cryopreservation, thawing the stem-cell seeded allograft containing the cell culture media to provide the bone augmentation composition for application to a bone treatment site. In one embodiment, a surviving stem cell population in the bone augmentation composition after thawing is between 50% and 90% of an initial stem cell population in the bone augmentation composition prior to freezing. In one embodiment, the seeding comprises a dynamic seeding where the hydrated allograft is agitated at least during application of the stem cell solution to the hydrated allograft; culturing the stem-cell seeded allograft for a predetermined (or limited time) to reduce stem cell attachment to the allograft. In one embodiment, the dynamic seeding, the limited time of culturing, and the freezing produce, after thawing, a surviving stem cell population in the bone augmentation composition that is between 50% and 90% of an initial stem cell population in the bone augmentation composition prior to freezing. In one embodiment, reducing attachment of the stem cells to the allograft fibers promotes a higher surviving stem cell population. In one embodiment, a prolonged time (as opposed to a limited time of culturing generally less than 5 hrs) reduces the surviving stem cell population. In one embodiment, the surviving stem cell population in the bone augmentation composition can range between 10% and 98%, and include the fractional percentages in between.

Statement 2. The method of statement 1, wherein the surviving stem cell population in the bone augmentation composition is between 1 and 10×10⁵ cells/ml, 1 and 7.5×10⁵ cells/ml, 1 and 5×10⁵ cells/ml, or 1 and 2.5×10⁵ cells/ml, and any intervening stem cell populations.

Statement 3. The method of any of the statements above, wherein the culturing the stem-cell seeded allograft for a limited time occurs for a period of about 0.5 to 5 hrs at a temperature of about 20 to 50° C.

Statement 4. The method of any of the statements above, wherein the culturing the stem-cell seeded allograft for a limited time occurs or a period of about 1 to 3 hrs at a temperature of about 20 to 50° C.

Statement 5. The method of any of the statements above, wherein the culturing the stem-cell seeded allograft for a limited time occurs for a period of about 1.5 to 2.5 hrs at a temperature of about 20 to 50° C.

Statement 6. The method of any of the statements above, wherein the dynamic seeding comprises shaking the hydrated allograft with the stem solution.

Statement 7. The method of any of the statements above, wherein the stem cell solution applied during the dynamic seeding has a stem cell density of about 1×10⁵ to 1×10⁶ cells/ml as counted by an Alamar Blue Assay.

Statement 8. The method of any of the statements above, wherein the stem cell solution applied during the dynamic seeding has a stem cell density of about 2×10⁵ to 8×10⁵ cells/ml as counted by an Alamar Blue Assay.

Statement 9. The method of any of the statements above, wherein the stem cell solution applied during the dynamic seeding has a stem cell density of about 5×10⁵ to 7×10⁵ cells/ml as counted by an Alamar Blue Assay.

Statement 10. The method of any of the statements above, wherein the freezing comprises storing the stem-cell seeded allograft containing the cell culture media at about −80° C.

Statement 11. The method of statement 10, wherein the freezing comprises utilizing a controlled freezing at a rate of about −0.5° C./min, about −1° C./min, about −2° C./min, about −5° C./min, and any intervening rates.

Statement 12. The method of statement 10, further comprising thawing at about room temperature to 50° C., or at about 37° C., or intervening temperatures.

Statement 13. The method of any of the statements above, wherein the cell culture media comprises a stem cell nourishment solution.

Statement 14. The method of any of the statements above, wherein the seeding and the culturing occur in a temperature range from room temperature to about 37° C., or from room temperature to about 50° C., or at any intervening temperature

Statement 15. The method of any of the statements above, wherein the seeding and the culturing produce a population of adherent stem cells in the allograft prior to freezing of about 5×10⁴ cells/ml to 1×10⁶ cells/ml as counted by an Alamar Blue Assay.

Statement 16. The method of any of the statements above, wherein the seeding comprises shaking or rotating the hydrated allograft while applying the stem cell solution.

Statement 17. The method of any of the statements above, wherein the shaking agitates the hydrated allograft at about 2 to 500 rpm.

Statement 18. The method of any of the statements above, wherein the shaking agitates the hydrated allograft at about 100 to 200 rpm.

Statement 19. The method of any of the statements above, wherein the shaking agitates the hydrated allograft at about 120 to 150 rpm.

Statement 20. The method of any of the statements above, wherein, while shaking the hydrated allograft, applying a solution having a concentration of about 1×10⁶ to 5×10⁶ stem cells/ml solution to the hydrated allograft.

Statement 21. The method of any of the statements above, further comprising freezing the hydrated allograft containing the stem cell solution at about −80° C., or at liquid nitrogen temperatures, or from −2 to −80° C., or at any intervening temperatures.

Statement 22. The method of statement 21, wherein the hydrated allograft containing the stem cell solution is frozen with dimethyl sulfoxide DMSO having a concentration less than about 20 weight percent, or less than about 10 weight percent, or less than about 5 weight percent, or less than about 2 weight percent or less than 1 weight percent, or less than 0.5 weight percent.

Statement 23. The method of any of the statements above, wherein the cortical fibers comprise an intertangled mixture of the cortical fibers.

Statement 24. A bone augmentation composition, comprising:

i) human mesenchymal stem cells derived from a source other than the patient being treated,

ii) a cell culture media. and

ii) an allograft comprising cortical fibers

wherein the allograft comprising the stem cells and the cell culture media is prepared using any of the methods of statements 1-23, and

wherein the allograft comprising the stem cells and the cell culture media, after freezing and thawing, has a surviving stem cell population in the bone augmentation composition between 1 and 10×10⁵ cells/ml, 1 and 7.5×10⁵ cells/ml, 1 and 5×10⁵ cells/ml, or 1 and 2.5×10⁵ cells/ml, and any intervening stem cell population, as counted by an Alamar Blue Assay.

Statement 25. The composition of statement 24, wherein the allograft comprising the stem cells and the cell culture media comprises a frozen composition stored in a container while awaiting application of the bone augmentation composition to a bone treatment site.

Statement 26. The composition of any of the statements 24-25, wherein the frozen composition comprises dimethyl sulfoxide DMSO as a cryo-preservative added to the composition prior to freezing, the cryo-preservative having a concentration less than about 20 weight percent, or less than about 10 weight percent, or less than about 5 weight percent, or less than about 2 weight percent or less than 1 weight percent, or less than 0.5 weight percent.

Statement 27. The composition of statement 26, wherein the frozen composition comprises a cell detachment solution, added to the composition prior to freezing, the cell detachment solution having a concentration less than about 20 weight percent, or less than about 10 weight percent, or less than about 5 weight percent, or less than about 2 weight percent or less than 1 weight percent, or less than 0.5 weight percent.

Statement 28. The composition of any of the statements 24-27, wherein the allograft comprises demineralized cortical fibers.

Statement 29. The composition of any of the statements 24-28, wherein the allograft comprises a loose, intertangled collection of the cortical fibers.

Statement 30. The composition of any of the statements 24-29, wherein the allograft comprising the stem cells and the cell culture media comprises a moldable product.

Statement 31. A bone augmentation composition, comprising:

i) stem cells,

ii) a cell culture media. and

ii) an allograft comprising bone fibers

wherein the allograft comprising the stem cells and the cell culture media, after freezing and thawing, has a surviving stem cell population in the bone augmentation composition between 1 and 5×10⁵ cells/ml, as counted by an Alamar Blue Assay.

Numerous modifications and variations of the present invention are possible in light of the above teachings. It is therefore to be understood that within the scope of the appended claims, the invention may be practiced otherwise than as specifically described herein.

1. A method for making a bone augmentation composition, comprising:
hydrating an allograft comprising cortical fibers with a cell culture media;
seeding the hydrated allograft with human mesenchymal stem cell solution derived from a source other than the patient being treated.
culturing the stem-cell seeded allograft for a predetermined time;
freezing the stem-cell seeded allograft containing the cell culture media with a cryopreservation solution; and
after cryopreservation. thawing the stem-cell seeded allograft containing the cell culture media to provide the bone augmentation composition for application to a bone treatment site,
wherein a surviving stem cell population in the bone augmentation composition after thawing is between 50% and 90% of an initial stem cell population in the bone augmentation composition prior to freezing.

2. The method of claim 1, wherein the surviving stem cell population in the bone augmentation composition is between 1 and 5×10⁵ cells/ml.

3. The method of claim 1, wherein the culturing the stem-cell seeded allograft for a limited time occurs for a period of about 0.5 to 5 hrs at a temperature of about 20 to 50° C.

4. The method of claim 1, wherein the seeding comprises shaking the hydrated allograft with the stem solution.

5. The method of claim 1, wherein the stem cell solution applied during the seeding has a stem cell density of about 1×10⁵ to 1×10⁶ cells/ml as counted by an Alamar Blue Assay.

6. The method of claim 1, wherein the freezing comprises storing the stem-cell seeded allograft containing the cell culture media at about −80° C.

7. The method of claim 1, wherein the cell culture media comprises a stem cell nourishment solution.

8. The method of claim 1, wherein the seeding comprises shaking or rotating the hydrated allograft while applying the stem cell solution.

9. The method of claim 8, wherein the shaking agitates the hydrated allograft at about 2 to 500 rpm.

10. The method of claim 1, wherein the hydrated allograft containing the stem cell solution is frozen with dimethyl sulfoxide DMSO having a concentration of less than about 10 weight percent.

11. The method of claim 1, wherein the cortical fibers comprise a loose, intertangled mixture of the cortical fibers.

12. A bone augmentation composition, comprising:
i) human mesenchymal stem cells derived from a source other than the patient being treated,
ii) a cell culture media. and
ii) an allograft comprising cortical fibers
wherein the allograft comprising the stem cells and the cell culture media, after freezing and thawing, has a surviving stem cell population in the bone augmentation composition between 1 and 5×10⁵ cells/ml, as counted by an Alamar Blue Assay.

13. The composition of claim 12, wherein the allograft comprising the stem cells and the cell culture media comprises a frozen composition stored in a container while awaiting application of the bone augmentation composition to a bone treatment site.

14. The composition of claim 13, wherein the frozen composition comprises dimethyl sulfoxide DMSO as a cryo-preservative added to the composition prior to freezing.

15. The composition of claim 12, wherein the allograft comprises demineralized cortical fibers.

16. The composition of claim 12, wherein the allograft comprises a loose intertangled collection of the cortical fibers.

17. The composition of claim 12, wherein the allograft comprising the stem cells and the cell culture media comprises a moldable product.

18. The composition of claim 12, wherein the allograft comprising the stem cells and the cell culture media is prepared using any of the methods of claims 1-11.

19. The composition of claim 12, wherein a surviving stem cell population in the bone augmentation composition after thawing is between 50% and 90% of an initial stem cell population in the bone augmentation composition prior to freezing.

20. A bone augmentation composition, comprising:
i) stem cells,
ii) a cell culture media. and
ii) an allograft comprising bone fibers
wherein the allograft comprising the stem cells and the cell culture media, after freezing and thawing, has a surviving stem cell population in the bone augmentation composition between 1 and 5×10⁵ cells/ml, as counted by an Alamar Blue Assay.
